(19) United States
(12) Patent Application Publication
(10) Pub. No.: US 2007/0259945 A1
(43) Pub. Date: Nov. 8, 2007

(54) METHOD FOR TREATING PAIN

(75) Inventors: Luciano De Petrocellis, Pozzuoli (IT); Sabatino Malone, Brusciano-Naples (IT); Vito De Novellis, S. Maria Capua Vetere (IT); Vincenzo Di Marzo, Pozzuoli (IT)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/549,334

(22) Filed: Oct. 13, 2006

Related U.S. Application Data
(60) Provisional application No. 60/729,114, filed on Oct. 19, 2005.

(51) Int. Cl. A61K 31/40; A61P 25/02; C07D 209/00

(52) U.S. Cl. 514/419; 548/452

(57) ABSTRACT

The present invention provides pharmaceutical compositions useful in a method for treating neuropathic pain, said method comprising administration of a pain-ameliorating effective amount of the compound according to formula I 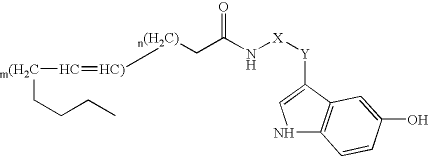
wherein X may be 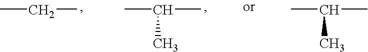
Y may be 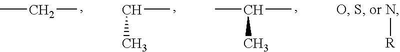
wherein R is H or (CH₂)ₘH, alternatively the bond between X and Y may be a double or triple bond, e.g. X═Y or X≡Y; wherein n is an integer of from 1 to 10, m is an integer of from 1 to 4 and the total carbon atoms in the alkenyl amide chain is from about 11 to about 20 as an active ingredient together with one or more pharmaceutically-acceptable additives, excipients or diluents.

### BACKGROUND OF THE INVENTION

1. Field of the Invention

The present invention relates to the treatment or prevention of pain or nociception.

2. Related Art

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. This range of sensations, as well as the variation in perception of pain by different individuals, renders a precise definition of pain difficult, however, many individuals suffer with severe and continuous pain.

Pain that is caused by damage to neural structures is often manifest as a neural supersensitivity or hyperalgesia and is termed “neuropathic” pain. Pain can also be “caused” by the stulation of nociceptive receptors and transmitted over intact neural pathways, such pain is termed “nociceptive” pain.

The level of stimulation at which pain becomes noted is referred to as the “pain threshold.” Analgesics are pharmaceutical agents which relieve pain by raising the pain threshold without a loss of consciousness. After administration of an analgesic drug a stimulus of greater intensity or longer duration is required before pain is experienced. In an individual suffering from hyperalgesia an analgesic drug may have an anti-hyperalgesic effect. In contrast to analgesics, agents such as local anaesthetics block transmission in peripheral nerve fibers thereby blocking awareness of pain. General anaesthetics, on the other hand, reduce the awareness of pain by producing a loss of consciousness.

### BRIEF SUMMARY OF THE INVENTION

It has now been discovered that certain compounds which exhibit the properties of blocking transient receptor potential vanilloid type 1 (TRPV1) channels and activating cannabinoid CB₁ receptors have a utility for the amelioration of pain and particularly for the amelioration of neuropathic pain.

Therefore, in one aspect, the method of the present invention utilizes analogues and/or homologues of the compound, N-arachidonoyl-serotonin (AA-5-HT) according to formula I 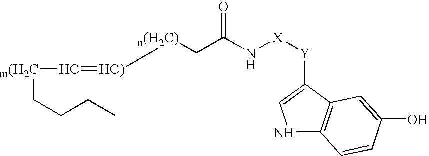
wherein X may be 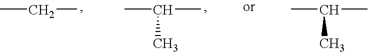
Y may be 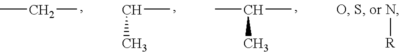
wherein R is H or (CH₂)ₘH, alternatively the bond between X and Y may be a double or triple bond, e.g. X═Y or X≡Y;
wherein n is an integer of from 1 to 10, m is an integer of from 1 to 4 and the total carbon atoms in the alkenyl amide chain is from about 11 to about 20 to ameliorate or treat pain.

The compounds of this invention may be prepared by reacting serotonin or an analogue or homologue thereof with a carboxylic acid to form the corresponding amide of said carboxylic acid and serotonin (or analogue or homologue thereof). This reaction may be carried out at conditions known in the art for preparing amides of fatty acids e.g., which fatty acids have similar reaction properties as the above carboxylic acids.

In another aspect, the invention provides a method for the treatment of pain using a compound in accord with formula I, the method comprising administering a pain-ameliorating effective amount of the compound.

In another embodiment, the method comprises administration of a pain-ameliorating effective amount of a compound according to formula I in the form of a pharmaceutical composition comprising a compound according to formula I as an active ingredient together with one or more pharmaceutically-acceptable additives.

In a further embodiment, the method comprises binding a compound according to formula I to the TRPV1 a warm-blooded animal, such as a human being, so as to beneficially inhibit the activity of said channel to activation by capsaicin, for example.

In a further embodiment, the method comprises binding a compound according to formula I to the fatty acid amide hydrolase of a warm-blooded animal, such as a human being, so as to enhance endocannabinoid levels and activate cannabinoid receptors in said animals to thereby ameliorate pain.

Yet other aspects of the invention are pharmaceutical compositions which contain the compound in accord with formula I and the use of the compound in accord with formula I for the preparation of medicaments and pharmaceutical compositions.

### DETAILED DESCRIPTION OF THE INVENTION

Genetic or pharmacological targeting of fatty acid amide hydrolase (FAAH), one of the enzymes catalysing endocannabinoid degradation, was shown to result in analgesic and anti-hyperalgesic actions that are due to the “indirect” activation (via enhancement of endocannabinoid levels) of cannabinoid CB₁ receptors. Additionally, genetic or pharmacological targeting of transient receptor potential vanilloid type 1 (TRPV1) channels was found to abolish thermal and inflammatory analgesia. We describe a class of “hybrid” FAAH inhibitors/TRPV1 antagonists with high efficacy against inflammatory hyperalgesia. These “hybrid” FAAH inhibitors are homolgues and/or analogues of AA-5-HT and have the general formula I: 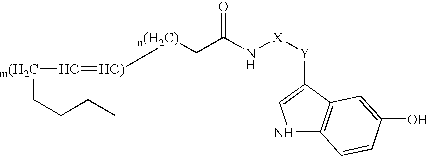
wherein X may be 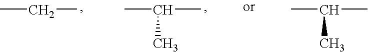
Y may be 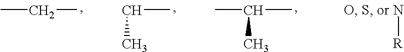
wherein R is H or (CH₂)ₘH, alternatively the bond between X and Y may be a double or triple bond, e.g. X═Y or X≡Y;
wherein n is an integer of from 1 to 10, m is an integer of from 1 to 4 and the total carbon atoms in the alkenyl amide chain is from about 11 to about 20 to ameliorate or treat pain;
N-arachidonoyl-serotonin (AA-5-HT) and its congeners inhibit FAAH as mixed inhibitors and in a range of concentrations between 1.5 and 15 μM depending of the animal species and type of enzyme preparation. We now show that the homologues and analogues of AA-5-HT described above in formula I, like AA-5-HT, also interact, by blocking their activation by capsaicin, with TRPV1 channels, whose gating plays a permissive role in the development of hyperlagesia, e.g. following formalin injection into the paw of laboratory animals. For AA-5-HT the IC₅₀ against capsaicin (100 nM) was calculated to be 130 nM (FIG. 1A), similar to that previously reported for the well known TRPV1 antagonist capsazepine (60 nM). AA-5-HT behaves as a non-competitive antagonist. Similar results are obtained with other congeners of PA-5-HT, provided that at least one double bond is present in the acyl chain.

When injected directly into the periaqueductal grey (PAG) of rats, AA-5-HT (4 μg/rat) potently inhibits both phases of the nociceptive response to formalin injected into the rat paw and concomitantly elevated anandamide levels in this area of the brainstem, when these are measured 20 min following injection. The anti-hyperalgesic effect is counteracted by the CB₁ receptor antagonist, AM251 (nmol/rat) and is occluded by the TRPV1 antagonist, capsazepine (6 nmol/rat). Thus, while not wishing to be bound by theory, it is believed that the compounds of formula 4, like AA-5-HT, ameliorate pain like the dual mechanism of action of AA-5-HT via both “indirect” activation of CB₁ and antagonism of TRPV1. The compound of formula I act at the supraspinal level by blocking the inhibitory effect of formalin on the OFF cells of the rostral ventromedial medulla, which receive synapses with cells from the PAG. Also this effect is reversed by AM251 and occluded by capsazepine. When injected into the paw, the compounds of formula I, like AA-5-HT selectively block the 2^{nd}, inflammatory phase of the nocifensive response to formalin, again in a way counteracted by AM251 and occluded by capsazepine, thus suggesting also a peripheral mode of action. The compounds of formula I are novel agents against anti-inflammatory pain, acting by enhancing endocannabinoid levels (via FAAH inhibition) and at the same time by antagonizing TRPV1.

The advantage of having in one molecule a FAAH inhibitor and a TRPV1 antagonist comes from the several experimental observations suggesting that FAAH inhibitors (i.e. “indirect” agonists of cannabinoid and fatty acid amide receptors) as well as direct cannabinoid receptor agonists (both CB1 and CB2) are very promising against inflammatory and neuropathic pain, and so are compounds that block TRPV1 receptors. However, different populations of neurons/cells and different mechanisms are involved in CB1/CB2- and TRPV1-mediated anti-inflammatory and anti-hyperalgesic/anti-allodynic effects. Therefore, if for example following nerve injury, only one of these different populations is destroyed, a compound only acting on that population will be ineffective, whereas a compound with “hybrid” activity will always be more effective. On the other hand if different nociceptive mechanisms cause pain, a drug targeting more of these mechanisms will be more efficacious than a drug specific for only one of them.

To use the compound of the invention or a pharmaceutically-acceptable salt thereof for the therapeutic treatment, which may include prophylactic treatment, of pain in mammals, which may be humans, the compound can be formulated in accordance with standard pharmaceutical practice as a pharmaceutical composition.

Suitable pharmaceutical compositions that contain the compounds of the invention may be administered in conventional ways, for example by oral, topical, parenteral, buccal, nasal, vaginal or rectal administration or by inhalation. For these purposes a compound of the invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions. A preferred route of administration is orally by tablet or capsule.

In addition to a compound of the present invention a pharmaceutical composition of this invention may also contain one or more other pharmacologically-active agents, or such pharmaceutical composition may be simultaneously or sequentially co-administered with one or more other pharmacologically-active agents.

Pharmaceutical compositions of this invention will normally be administered so that a pain-ameliorating effective daily dose is received by the subject. The daily dose may be given in divided doses as necessary, the precise amount of the compound received and the route of administration depending on the weight, age and sex of the patient being treated and on the particular disease condition being treated according to principles known in the art. A preferred dosage regime is once daily.

A further embodiment of the invention provides a pharmaceutical composition which contains a compound of the invention as defined herein or a pharmaceutically-acceptable salt thereof, in association with a pharmaceutically-acceptable additive such as an excipient or carrier.

A yet further embodiment of the invention provide the use of the compound of the invention, or a pharmaceutically-acceptable salt thereof in the manufacture of a medicament useful for blocking the TRPV1 channel in a warm-blooded animal such as a human being.

Still another embodiment of the invention provides a method of binding the compound of the invention to the TRPV1 channel of a warm-blooded animal, such as a human being, in need of treatment for pain, which method comprises administering to said animal an effective amount of a compound of formula I or a pharmaceutically-acceptable salt thereof.

A yet further embodiment of the invention comprises the use of the compound of the invention, or a pharmaceutically-acceptable salt thereof in the manufacture of a medicament useful for indirectly activating the cannabinoid CB₁ receptor in a warm-blooded animal such as a human being.

Still another embodiment of the invention provides a method of binding the compound of the invention to the fatty acid amide hydrolase of a warm-blooded animal, such as a human being, in need of treatment for pain, which method comprises administering to said animal an effective amount of a compound of formula I or a pharmaceutically-acceptable salt thereof.

The foregoing description details specific methods and compositions that can be employed to practice the present invention, and represents the best mode contemplated. Thus, however detailed the foregoing may appear in text, it should not be construed as limiting the overall scope hereof rather, the ambit of the present invention is to be governed only by the lawful construction of the appended claims.

What is claimed is:

1. A pharmaceutical composition comprising a pain-ameliorating effective amount of a compound according to formula I 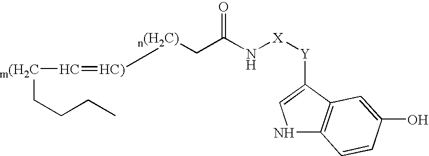
wherein X may be 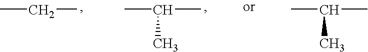
Y may be 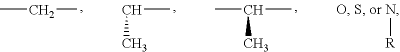
wherein R is H or (CH₂)ₘH, alternatively the bond between X and Y may be a double or triple bond;
wherein n is an integer of from 1 to 10, m is an integer of from 1 to 4 and the total carbon atoms in the alkenyl amide chain is from about 11 to about 20, as an active ingredient together with one or more pharmaceutically-acceptable additives, excipients or diluents.

2. A method for treating neuropatic pain, said method comprising administration of a pain-ameliorating effective amount of the compound according to formula I 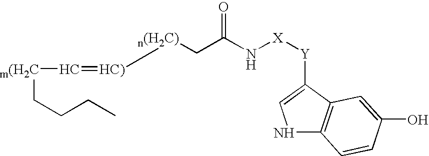
wherein X may be 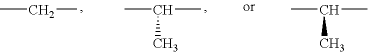
Y may be 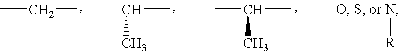
wherein R is H or (CH₂)ₘH, alternatively the bond between X and Y may be a double or triple bond;
wherein n is an integer of from 1 to 10, m is an integer of from 1 to 4 and the total carbon atoms in the alkenyl amide chain is from about 11 to about 20.

3. A method for treating neuropathic pain, said method comprising administration of a pain-ameliorating effective amount of a pharmaceutical composition according to claim 1.

4. A method comprising binding a compound according to formula I 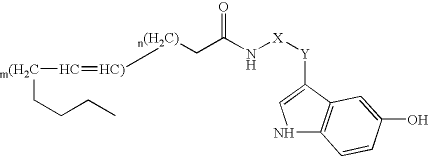
wherein X may be 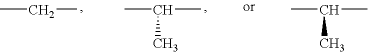
Y may be 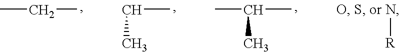
wherein R is H or (CH₂)ₘH, alternatively the bond between X and Y may be a double or triple bond;
wherein n is an integer of from 1 to 10, m is an integer of from 1 to 4 and the total carbon atoms in the alkenyl amide chain is from about 11 to about 20 to the TRPV1 channel of a warm-blooded animal, such as a human being, so as to beneficially inhibit the activity of said channel to thereby ameliorate pain.

5. A method comprising binding a compound according to formula I 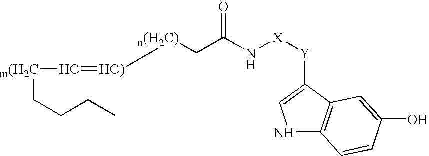
wherein X may be 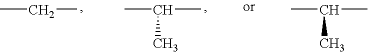
Y may be 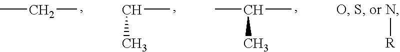
wherein R is H or (CH₂)ₘH, alternatively the bond between X and Y may be a double or triple bond;
wherein n is an integer of from 1 to 10, m is an integer of from 1 to 4 and the total carbon atoms in the alkenyl amide chain is from about 11 to about 20 to the fatty acid amide hydrolase of a warm-blooded animal, such as a human being, so as to activate said receptor to enhance endocannabinoid levels and activate cannabinoid receptors in said animal to thereby ameliorate pain.

6. A method comprising binding a compound according to formula I 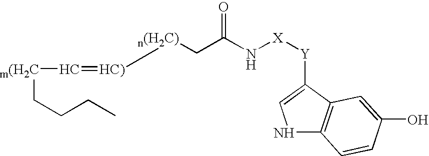
wherein X may be 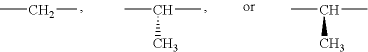
Y may be 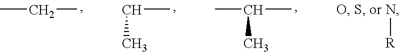
wherein R is H or (CH₂)ₘH, alternatively the bond between X and Y may be a double or triple bond;
wherein n is an integer of from 1 to 10, m is an integer of from 1 to 4 and the total carbon atoms in the alkenyl amide chain is from about 11 to about 20 to the fatty acid amide hydrolase and the TRPV1 channel of a warm-blooded animal, such as a human being so as to enhance endocannabinoid levels, activate cannabinoid receptors and beneficially inhibit the activity of said channel to enhance endocannabinoid levels in said animal to thereby ameliorate pain.
